(19) United States
(12) Patent Application Publication
(10) Pub. No.: US 2021/0346292 A1
(43) Pub. Date: Nov. 11, 2021

(54) Pharmaceutical preparation for increasing stability and bioavailability of Botulinum toxin A and its complex

(71) Applicants: Bal Ram Singh, North Dartmouth, MA (US); Raj Kumar, North Dartmouth, MA (US); Boke Zhang, Brighton, MA (US)

(72) Inventors: Bal Ram Singh, North Dartmouth, MA (US); Raj Kumar, North Dartmouth, MA (US); Boke Zhang, Brighton, MA (US)

(73) Assignee: Prime Bio, Inc., Dartmouth, MA (US)

(21) Appl. No.: 16/867,693

(22) Filed: May 6, 2020

(51) Int. Cl. A61K 9/127; A61K 9/107; A61K 9/51

(52) U.S. Cl. CPC A61K 9/127; A61K 45/06; A61K 9/5146; A61K 9/107

(57) ABSTRACT

The main aspect of present invention is to provide a pharmaceutical composition to increase the stability of liquid formulation of botulinum toxin and related proteins. The present invention provides a method to stabilize toxin in liquid formulation. Lipid based drug delivery system is known to increase the bioavailability of drugs (Amidon et al., 1995; Jannin et al., 2008). We investigated the stability of BoNT/A toxin and complex. We used two formulations in liquid phase: combination of lipids and liposomes, with two different storage conditions: 4° C. and 25° C. The present invention also provides a method for efficient delivery of botulinum toxin through skin as a topical application.

### BACKGROUND OF THE INVENTION

Botulinum toxins (BoNTs) consist of a primarily two major domains: binding domain (Heavy chain (HC)) and a catalytic domain (Light chain (LC)), linked through a disulfide bond (Montecucco and Schiavo 1995). Upon binding specifically to the presynaptic nerve membrane, BoNT is internalized through endocytosis, and the LC is translocated through a membrane pore formed by the translocation domain (TD) of the HC (Li and Singh 2000). The LC is a zinc-metalloprotease and acts as an endopeptidase with remarkable substrate specificity requiring a substantially long peptide sequence, depending on the serotype (Segelke et al. 2004). This is unique to BoNTs as other microbial metalloproteases can recognize sequences as short as a dipeptide (Segelke et al., 2004; Silvaggi et al. 2007). Serotype A cleaves SNAP-25 (25-kDa synaptosome associated protein) and the light chain of BoNT/A will be examined in this paper (Li and Singh 1999; Kukreja and Singh 2007).

BoNTs are secreted from the Clostridium botulinum bacteria in the form of multimeric complexes, with a set of non-toxic proteins coded for by genes adjacent to the neurotoxin gene (Inoue et al., 1996; Singh et al., 2014). Botulinum complex size ranges from 300 kDa to 900 kDa and exist in three progenitor toxin forms: M (medium), L (large) and LL (extra-large) forms. The M form consists of neurotoxin (150 kDa) and a nontoxic protein component (120 kDa) which is called neurotoxin binding protein (NBP) (Singh et al., 1995) or nontoxic non-hemagglutinin component (NTNH) (East and Collins, 1994) with 12S molecular size (the molecular size of complex forms is expressed as sedimentation equilibrium values). The L form has molecular weight of about 500 kDa and a molecular size of 16S. The LL form is about 900 kDa and 19S.

Currently major BoNT therapeutic products include BoNT/A complex (marketed as Botox® and Dysport®), BoNT/B complex (marketed as Myobloc® and Neurobloc®), and isolated BoNT/A without NAPs (marketed as Xeomin®). Although there is no therapeutic role defined for NAPs, these may play a role in the stability of the BoNT formulation and in diffusion of the injected BoNT for therapeutic purposes (Carli et al., 2009; Shone et al., 2011). In BoNT/A complex preparations adding either sodium chloride (Botox®, Allergan, Inc.) or lactose (Dysport®, Ipsen, Ltd.) protect the steric conformation of BoNT (Panicker and Muthane, 2003). Human serum albumin is also added to prevent loss from surface adsorption. The toxin is then dried either with freezing (Dysport®) or without freezing (Botox®, Allergan, Inc.) (Panicker and Muthane, 2003). These as well as the pure BoNT/A product, Xeomin®, are lyophilized products which are reconstituted with saline solution maintained near physiological pH.

The botulinum toxin type B product (Myobloc®, Neuroblock®) is provided in liquid form at pH 5.6, as opposed to a lyophilized powder that requires reconstitution in saline. It nevertheless is also based on the complex of BoNT/B neurotoxin and NAPs. BoNT/B has shown stability for months when stored appropriately at 2° C. to 8° C., whereas BoNT/A must be stored at −5° C. as a powder and must be used within hours once reconstituted according to the manufacturer's recommendation (Kim et al., 2003).

The present invention provides a method to stabilize toxin in liquid formulation. Lipid based drug delivery system is known to increase the bioavailability of drugs (Amidon et al., 1995; Jannin et al., 2008). We investigated the stability of BoNT/A toxin and complex. We used two formulations in liquid phase: combination of lipids and liposomes, with two different storage conditions: 4° C. and 25° C. The present invention also provides a method for efficient delivery of botulinum toxin through skin as a topical application.

### BRIEF SUMMARY OF THE INVENTION

The main aspect of present invention is to provide a pharmaceutical composition to increase the stability of liquid formulation of botulinum toxin and related proteins.
Another aspect of present invention is the use of lipids.
Another aspect of present invention is the use of lipids in certain ratios.
Another aspect of present invention is the use of different herbal lipids.
Another aspect of the present invention is the lipids are DOTAP (1,2-dioleolyl-3-triethylammonium-propane), DPPC (Dipalmitoylphosphatidylcholine), and cholesterol.
Another aspect of the present invention is the ratio of DOTAP:DPPC:Cholesterol is 5:5:3.
Another aspect of the present invention is the mixing of the lipids with DOTAP/glutaryl PE 99:1 mol/mol) and non-ionic amphiphiles or detergents such as Tween 80 or SPAN-80 in chloroform or chloroform/water mixture.
Another aspect the formation of liposomes.
Another aspect of the present invention is the encapsulation of protein in the liposome.
Another aspect of the present invention is the encapsulated proteins are botulinum toxin A and botulinum toxin complex A.
Another aspect of the present invention is the method of encapsulation of toxin or complex.
a. Formation of lipid film;
b. Resuspend the lipid film in solution A and Solution B. Solution A (Botulinum toxin A+10 mM Sodium Phosphate buffer, pH=7.1), and solution B (Botulinum toxin complex A+10 mM Sodium Phosphate buffer, pH=7.1);
c. After resuspension, several freeze-thaw cycle was performed;
d. Sonicate the resuspended lipids;
e. Centrifuge at 4° C. using spin column several times.
   Another aspect of the present invention is the formulation preparation in a form of emulsion, mixture, or suspension.
   Another aspect of the present invention is the use of preservatives in liposomes, emulsion, mixture, or suspension including phenoxyethanol.
   Another aspect of the present invention is the use of lubricants such as sodium hyaluronate.
   Another aspect of the present invention is the endopeptidase activity of encapsulated proteins.
   Another aspect of the present invention is the pharmaceutical composition will be with excipients such as oil solution, mixed glycerides, water-soluble co-solvents and/or surfactants (such as hydrogenated castor oils),
   Another aspect of the present invention is the pharmaceutical composition may have excipients such as humectants (glycerin, lecithin or propylene glycol) and emolliants (zinc oxide, white petrolatum, dimethicone, lanolin, etc.).
   Another aspect of present invention, the pharmaceutical composition further comprises surface active agents, chelating agents, salicylates, anti-inflammatory agents, antibacterial agents, antifungal, anti-viral agents or phenothiazines.
   Another aspect of the invention, the pharmaceutical composition further comprises of other associated proteins of botulinum toxin complex.
   Another aspect of the invention, the pharmaceutical composition further comprises of liposomes mixed with retinoids, alpha hydroxyl acids and allantonin.
   Another aspect of the invention, the pharmaceutical composition further comprises of other Human Serum Albumin or IgG.
   Another aspect of the invention, the pharmaceutical composition further comprises of other proteins of Clostridium botulinum.
   Another aspect of the present invention is the pharmaceutical composition is a lyophilized or gel form.
   Another aspect the pharmaceutical composition is stabilized at a pH in between 5.5 and 8.0.

### DETAILED DESCRIPTION OF THE INVENTION

Described herein are formulations, pharmaceutical formulations, and methods of preparing and using the stable formulations described herein. These pharmaceutical formulations may be prepared by the processes described herein. In some variations the therapeutic agent is botulinum toxin A and botulinum toxin complex A.

In some variations the pharmaceutical formulations described herein can be used for the treatment, prevention, inhibition, delaying onset of, or causing regression of one or more neuromuscular diseases and conditions. In some variations the diseases or conditions include neuronal regeneration/sprouting, disease involving muscle movement, various wounds, scars and gastrointestinal symptoms.

Botulinum neurotoxin is a large protein toxin (approximately 150 kDa) that is able to bind and internalize to motor neurons very specifically. BoNTs are produced by Clostridium botulinum along with several neurotoxins associated polypeptides (NAPs). The toxin with NAPs is termed as complex toxin. Present application provides the method to have a stable liquid formulation. The method used in this application tested three different conditions; a) stability of the formulation in the presence of lipids, b) stability of the formulation in the encapsulated liposome, c) stability of the formulation in emulsion, mixture, or suspension form, and d) stability of the formulation in cellular model (function of the main active therapeutic ingredient). The present invention provides a pharmaceutical formulation of toxin with lipids.

In the first part of the present application, a lipid solution was made by dissolving a lipid film, made of Dotap: DPCC: Cholesterol (5:5:3), in 10 mM sodium phosphate buffer, pH 7.1. Dissolve the therapeutic proteins, BoNT/a toxin and BoNT/a toxin complex, in the solution of lipids. Activity was performed at different time points after incubating solution at 4° C. and 25° C. Activity of the enzyme was performed against the full-length substrate. Prior to activity reaction, lipid solution, containing enzyme, was incubated with the reaction buffer, 10 mM sodium phosphate (pH 7.4) containing 150 mM NaCl and 1.25 mM DTT (dithiotritol) for 30 mins at 37° C. After incubation proteins were incubated with substrate for 1 hr at 37° C. Reaction was stopped by adding 4×SDS-sample buffer. At 4° C., liquid formulation of both toxin and complex in lipids holds their endopeptidase activity for 8 weeks, as assessed by endopeptidase activity. After 8 weeks, 90% of activity remains for the toxin whereas complex hold 100% of its activity. After 24 weeks, the complex still holds about 57% of its activity, whereas toxin holds 29% of its endopeptidase activity (FIG. 1).

In the second part, lipid film was resuspended in the buffer, mM sodium phosphate, pH 7.1, containing toxin or complex. Encapsulated liposomes were formed by several freeze-thaw cycle followed by sonication. Unencapsualted proteins were removed from the supernatant using spin columns. The activity of encapsulated proteins were determined as follows. Prior to activity reaction, encapsulated proteins were incubated in the reaction buffer for 30 mins at 37° C. After incubation proteins were incubated with the substrate for 1 hr at 37° C. Reaction was stopped by adding 4×SDS-sample buffer. For botulinum toxin samples, reactions were performed in 10 mM sodium phosphate buffer, pH 7.1, containing 1.25 mM DTT and 0.2% Triton X-100. Whereas for botulinum toxin complex samples, reactions were performed 10 mM sodium phosphate, pH 7.1, containing 150 mM NaCl, 1.25 mM DTT and 0.2% Triton X-100. At 4° C., liquid formulation of both toxin and complex in lipids holds their endopeptidase activity for 8 weeks as assessed by endopeptidase activity (FIG. 2).

In the third part, proteins were emulsified or suspended with encapsulated microspheres and nanoshperes (nanoparticles) containing propylene glycol (0.3-6%), phenoxyethanol (0.1-5%), sodium hyaluronate (0.01-1%), caprylic/capric triglyceride (0.5-10%), hydrogenated castor oil (1-15%) and span-80 (0.01-6%) in water Protein was emuslsified by rotating the solution at room temperature (25° C.) for 15 min. Activity of emulsified protein was determined as above (FIG. 3).

In the fourth part, encapsulation or emulsification of protein was performed by using the similar procedure as above. Liposome incubated proteins are dissolved in the serum free media and incubated with M-17 neuroblastoma cells for 48 hrs. After incubation times, cells were detached and lysed using M-per reagent (Thermo Fisher Scientific). SNAP-25 cleavage in M-17 cells wasmonitored using western blot. Anti-SNAP25 monoclonal antibody was used as a primary antibody, and anti-rabbit IgG alkaline phosphatase was used as a secondary antibody for western blot. The blot was developed using BCIP (5-bromo-4-chloro-3-indolylphosphate toluidine; Sigma Aldrich) reagent. The Western blot image showed that both encapsulated toxin and complex had higher activity than the unencapsulated proteins (FIG. 4), indicating better delivery of encapsulated toxin inside the M17 cells. Emulsified formulation is not good for cell morphology that's why cellular assay was not performed in this formulation.

### FIGURE DESCRIPTIONS

FIG. 1: Stability data of Botulinum toxin A and its complex in lipid mixture solution (DOTAP: DPCC: Cholesterol:: 5:5:3). Stability experiments were performed in two conditions; 4° C. and ° C. All the reactions were performed at 37° C. in the 10 mM sodium phosphate buffer, pH 7.4, 150 mM NaCl and 1.25 mM DTT. Prior to reaction, protein solutions were incubated at 37° C. for 30 mins in the reaction buffer.

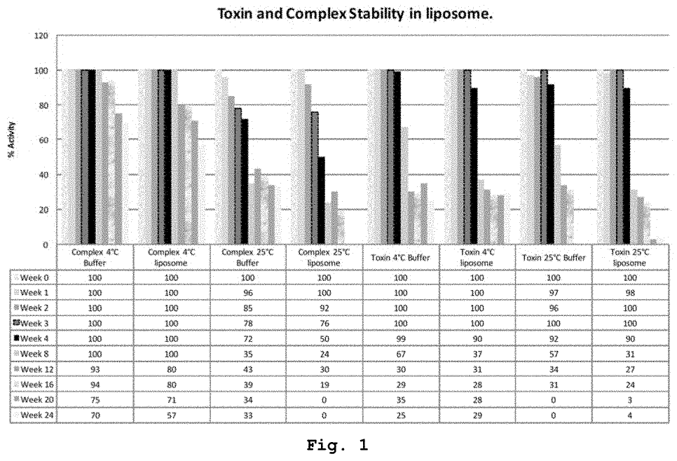

FIG. 2: Stability data of liposome encapsulated Botulinum toxin A and its complex. Stability experiments were performed in two conditions; 4° C. and 25° C. All the reactions of liposome encapsulated botulinum toxin A were performed at 37° C. in 10 mM sodium phosphate buffer, pH 7.1, containing 1.25 mM DTT and 0.2% Triton X-100. All the reactions of liposome encapsulated botulinum toxin complex A were performed at 37° C. in 10 mM sodium phosphate buffer, pH 7.1, containing 150 mM NaCl, 1.25 mM DTT and 0.2% Triton X-100. Prior to reaction, protein solutions were incubated at 37° C. for 30 mins in the reaction buffer.

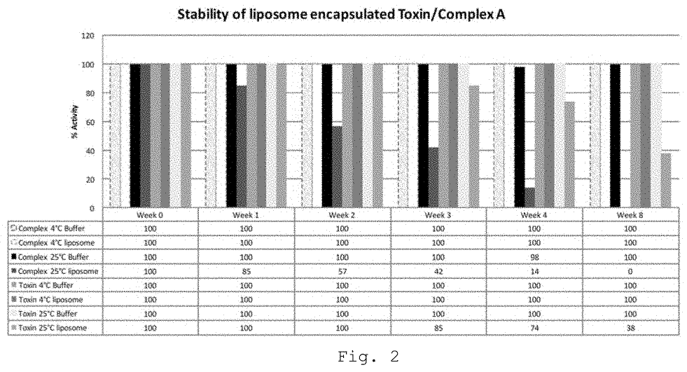

FIG. 3A and FIG. 3B: Stability data of Botulinum toxin A and its complex in lipid mixture solution in emulsified nanosphere formulations. The stability data of two different nanosphere formulations i.e. HA1 and HA2 are depicted in FIG. 3A and FIG. 3b respectively. Initially, stability experiments were performed in two conditions; 4° C. and 25° C. All the reactions were performed at 37° C. in the 10 mM sodium phosphate buffer, pH 7.4, 150 mM NaCl and 1.25 mM DTT. Prior to reaction, protein solutions were incubated at 37° C. for 30 mins in the reaction buffer. After month 5, samples were divided into two parts and store at −20° C. and −80° C. 13th month stability data was of samples stored at −20° C. and −80° C. A) Weekly stability data of HA1 and HA2. B)4th, 5th and 13th month stability data.

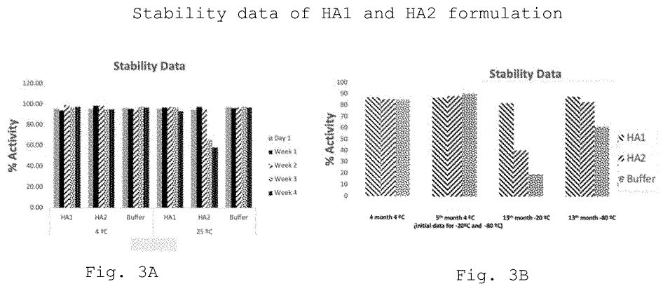

FIG. 4: Western blot of the cleavage of SNAP-25. Lane 1: Marker, lane 2: control M-17 cells without any treatment, lane 3: encapsulated botulinum toxin complex A treated M-17 cells, lane 4: botulinum toxin complex A (unencapsulated) treated M-17 cells, lane 5: encapsulated botulinum toxin A treated M-17 cells, and lane 6: botulinum toxin (unencapsulated) A treated M-17 cells. Samples for Western blot were prepared after 48 hr of incubation at 37° C. U and C are uncleaved SNAP-25 and cleaved SNAP-27.

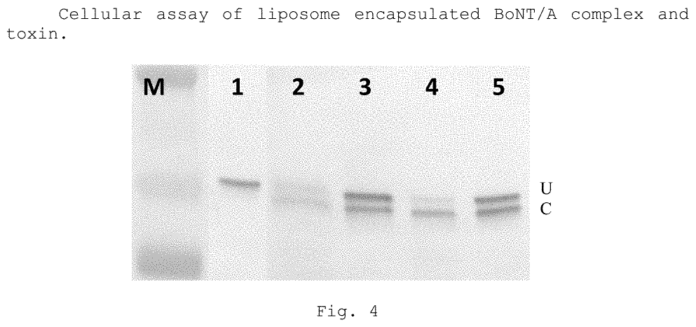

### REFERENCES

1. Montecucco C., and Schiavo, G. (1995). Structure and function of tetanus and botulinum neurotoxins. Q Rev Biophys., 28, 423-472.
2. Li L, and Singh B. R. (2000). Spectroscopic analysis of pH induced changes in the molecular features of type A botulinum neurotoxin light chain. Biochemistry, 39, 6466-6474.
3. Segelke B., Knapp M., Kadkhodayan S., Balhorn R., and Rupp B. (2004). Crystal structure of Clostridium botulinumneurotoxin protease in a product-bound state: Evidence for noncanonical zinc protease activity. PNAS, 101, 6888-6893.
4. Silvaggi N., Boldt, G. E., Hixon M. S., Kennedy J. P., Tzpori S., Janda K. D., and Allen K. N. (2007) Structures of Clostridium botulinum Neurotoxin Serotype A Light Chain Complexed with Small-Molecule Inhibitors Highlight ActiveSite Flexibility. Chemistry and Biology, 14, 533-542.
5. Li L., and Singh B. R. (1999). High-level expression, purification, and characterization of recombinant type A botulinum neurotoxin light chain. Protein Expr Purif. 17, 339-44
6. Kukreja R. V., and Singh B. R. (2007Comparative role of neurotoxin-associated proteins in the structural stability and endopeptidase activity of botulinum neurotoxin complex types A and E.). Biochemistry, 46, 14316-24.
7. Inoue K, Fujinaga Y, Watanabe T et al. Molecular composition of Clostridium botulinum type A progenitor toxins. Infect. Immun. 1996, 64, 1589-1594.
8. Singh B R, Chang T W, Kukreja R and Cai S. The Botulinum Neurotoxin Complex and the Role of Ancillary Proteins. In: Molecular Aspects of Botulinum Neurotoxin (Foster, Keith. A., Ed.), Springer, New York. Pp. 2014, 69-102.
9. Singh B R, Foley J, and Lafontaine C. Physico-chemical characterization of the botulinum neurotoxin binding protein from type E botulinum producing Clostridium botulinum. J. Protein Chem 1995, 14, 7-18.
10. East A K, and Collins M D. Conserved structure of genes encoding components of the botulinum neurotoxin complex M and the sequence of the gene encoding for the nontoxic component in nonproteolytic Clostridium botulinum type F. Curr Microbiol 1994, 29, 69-77.
11. Carli L, Montecucco C, and Rossetto O. Assay of diffusion of different botulinumneurotoxin type A formulations injected in the mouse leg. Muscle Nerve 2009. 40, 374-380.
12. Stone H F, Zhu Z, Thach T Q, and Ruegg C L. Characterization of diffusion and duration of action of a new botulinum toxin type A formulation. Toxicon 2011, 58, 159-167.
13. Panicker J N, and Muthane J B. Botulinum toxins: Pharmacology and its current therapeutic evidence for use. Neurol India 2003, 51, 455-460.
14. Kim E J, Ramirez A L, Reeck J B, and Maas C S. The role of botulinum toxin type B (Myobloc) in the treatment of hyperkinetic facial lines. PlastReconstr Surg. 2003, 112, 88S93S; discussion 94S-97S.
15. G. L. Amidon, H. Lennernas, V. P. Shah, J. R. Crison. A theoretical basis for a biopharmaceutic drug classification: the correlation in vitro drug product dissolution and in vivo bioavailability. Pharm Res, 12 (1995), 413-420.
16. V. Jannin, J. Musakhanian, D. Marchaud. Approaches for the development of solid and semi-solid lipid-based formulations. Adv Drug Deliv Rev, 60 (2008), 734-746.

What is claimed is:

1. A pharmaceutical composition comprising proteins with lipids, solution as emulsion or suspension, and/or encapsulated microspheres including liposomes.

2. The pharmaceutical composition as claimed in claim 1, wherein the proteins are botulinum toxin or complex thereof or various associated proteins of botulinum toxin.

3. The pharmaceutical composition as claimed in claim 1, wherein the pharmaceutical composition further comprises proteins of Clostridium botulinum.

4. The pharmaceutical composition as claimed in claim 1, wherein the pharmaceutical composition comprises therapeutic protein including various serotypes of botulinum toxin, vaccines and protein hormones.

5. The pharmaceutical composition as claimed in claim 1, wherein the lipids are charged with one of positive or negative molecules, or the lipids are neutral molecules.

6. The pharmaceutical composition as claimed in claim 1, wherein the lipids are mixture of charged and neutral lipids.

7. The pharmaceutical composition as claimed in claim 1, wherein the lipids are herbal or plant lipids.

8. The pharmaceutical composition as claimed in claim 1 further comprising non-ionic amphiphiles or detergents including one of glutaryl PE, Tween 80, Tween 60, Tween 20, PEGs, Cremophor EL or SPAN 80, or combination thereof to be mixed with the lipids.

9. The pharmaceutical composition as claimed in claim 1, wherein the emulsion is encapsulated microspheres and nanospheres (nanoparticles) containing propylene glycol (0.3-6%), phenoxyethanol (0.1-5%), sodium hyaluronate (0.01-1%), caprylic/capric triglyceride (0.5-10%), hydrogenated castor oil (1-15%) and Span-80 (0.01-6%) with water.

10. The pharmaceutical composition as claimed in claim 1, wherein the lipids are one of microspheres or nanospheres, including nanoparticle solution.

11. The pharmaceutical composition as claimed in claim 1, wherein the lipids are composed of oil solutions including for example, triglyceride, ethyl icosapentate, castor oil, tocopherol nicotinate, teprenone, indomethacin franesil, soy-bean oil, tea oil, sunflower seed oil, vegetable oil, fish oil, sesame oil, soy-bean oil, tea oil, sunflower seed oil, vegetable oil, fish oil, sesame oil, soy-bean oil, tea oil, sunflower seed oil, vegetable oil, fish oil, sesame oil, soy-bean oil, tea oil, sunflower seed oil, vegetable oil, fish oil, sesame oil, Labrafac Lipophile WL 1349 oil, and dronabinol.

12. The pharmaceutical composition as claimed in claim 1, wherein the pharmaceutical composition to be with excipients including oil solution, mixed glycerides, water-soluble co-solvents and surfactants, including Permulen TR1, Permulen TR2, RH-40, Tween-80, Tween-60, etc. and self-made creams.

13. The pharmaceutical composition as claimed in claim 1, wherein the pharmaceutical composition further comprises surface active agents, chelating agents, salicylates, anti-inflammatory agents, antibacterial agents, antifungal agents, antiviral agents or phenothiazines and bioactive peptides including pentapeptide KTTKS, tetrapeptide GQPR, hexapeptide argireline, tripeptide GHK, Snap-8 octapeptide and oligo-peptides.

14. The pharmaceutical composition as claimed in claim 1, wherein the pharmaceutical composition further comprises humectants including propylene glycol or lecithin; and emolliants including zinc oxide or dimethicone.

15. The pharmaceutical composition as claimed in claim 1, wherein the pharmaceutical composition further comprises a stabilizer including human serum albumin or IgG.

16. The pharmaceutical composition as claimed in claim 1, wherein the pharmaceutical composition is a lyophilized powder, lotion, serum or gel.

17. The pharmaceutical composition as claimed in claim 1, wherein the pharmaceutical composition is an encapsulated liposome or emulsified proteins with retinoids, alpha hydroxyl acids, hyaluronic acid and/or sodium salt, resveratrol, stem cells, EGFs (epidermal growth factors), KGFs (keratinocyte growth factors), FGFs (fibroblast growth factors), HGH (human growth hormones), niacinamide, aloe vera, allantoin and therapeutic agents thereof.

18. The pharmaceutical composition as claimed in claim 1, wherein the pharmaceutical composition is stabilized at a pH in between 5.5 and 8.0.

19. A method of treatment administrating effective amount of pharmaceutical composition comprising proteins with lipids, solution as emulsion or suspension, and/or encapsulated microspheres including liposomes.

20. The method claimed in claim 19, wherein the method of administration route includes one of topical, intranasal, injectable and oral, wherein the administration is to treat local and systemic conditions including neuromuscular, gastrointestinal, diabetic, cardiovascular, reproductive issues and skin problems.
